(19) United States
(12) Patent Application Publication
(10) Pub. No.: US 2004/0138694 A1
(43) Pub. Date: Jul. 15, 2004

(54) Intravascular filtering membrane and method of making an embolic protection filter device

(75) Inventors: The Thomas Trinh Tran, Coon Rapids, MN (US); Mark S. Smith, Coon Rapids, MN (US); Horng-Ban Lin, Maple Grove, MN (US); Narin Anderson, Savage, MN (US); Justin M. Crank, St. Louis Park, MN (US); James G. Hansen, Coon Rapids, MN (US); Paul J. Miller, Vadnais Heights, MN (US); Steven M. Spencer, Minneapolis, MN (US); Richard J. Traxler, Minneapolis, MN (US)

Correspondence Address: David M. Crompton, CROMPTON, SEAGER & TUFTE, LLC, Suite 895, 331 Second Avenue, Minneapolis, MN, 55401-2246, US

(73) Assignee: SciMed Life Systems, Inc.

(21) Appl. No.: 10/342,546

(22) Filed: Jan. 15, 2003

(51) Int. Cl. A61M 29/00

(52) U.S. Cl. 606/200

(57) ABSTRACT

An embolic protection filter device having a filtering membrane, an integrally formed waist, and a flexible hoop affixed to the filtering membrane by melt bonding. A method of making filtering membranes for an embolic protection filter device comprising blow molding the filtering membranes. A method of making an embolic protection filter device comprising blow molding a filtering membrane and simultaneously melt bonding a flexible hoop to the filtering membrane.

### FIELD OF THE INVENTION

This invention pertains to intravascular medical devices for embolic protection. More particularly, the present invention pertains to embolic protection filters and methods of making the same.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

### BRIEF SUMMARY

An example embodiment pertains to an embolic protection filter device. The embolic protection filter device may have an elongate filtering membrane having an integrally formed waist. The filtering membrane may be heat bonded or melt bonded to a flexible supporting member.

Another example embodiment pertains to a method of making shaped filtering membranes for embolic protection filter devices by blow molding or vacuum molding an extruded or otherwise formed polymeric tube to the desired shape and thickness.

Another example embodiment pertains to a method of making an embolic protection filter device. A polymeric tube may be extruded, shaped by material removal or selective heating and stretching and blow molded into a filtering membrane shape. A hoop or a hoop and strut apparatus may be inserted into the blow molding apparatus and the blow molding process may simultaneously shape the filtering membrane and affix it to the hoop.

### BRIEF DESCRIPTION OF THE DRAWINGS

FIG. 1 is a perspective view of an example embolic protection filter device;

FIG. 2a is a perspective view of an example moldable tube suitable for use in making one or more embolic protection filter devices;

FIG. 2b is a perspective view of another example moldable tube suitable for use in making one or more embolic protection filter devices;

FIG. 3a is a perspective view of an example wire frame and strut assembly suitable for use in making one or more embolic filter protection devices;

FIG. 3b is a front view of the example wire frame and strut assembly of FIG. 3a;

FIG. 3c is an end view of the example wire frame and strut assembly of FIG. 3a;

FIG. 4 is a front section view of a portion of a blow molding apparatus suitable for use in making one or more embolic filter protection devices;

FIG. 5a is a front section view of a central portion of a blow molding apparatus suitable for use in making one or more embolic filter protection devices which also depicts a hoop and strut assembly within the mold;

FIG. 5b is a front section view of a central portion of a blow molding apparatus suitable for use in making one or more embolic filter protection devices which also depicts two hoop assemblies within the mold;

FIG. 6 is a perspective view of an example molded tube and wire frame apparatus suitable for use in making one or more embolic filter protection devices; and

FIG. 7 is a perspective view of a filter membrane made from the molded tube of FIG. 6 by cutting the tube into two portions, each portion forming an embolic protection filter device.

### DETAILED DESCRIPTION

The following description should be read with reference to the drawings, wherein like reference numerals indicate like elements throughout the several views.

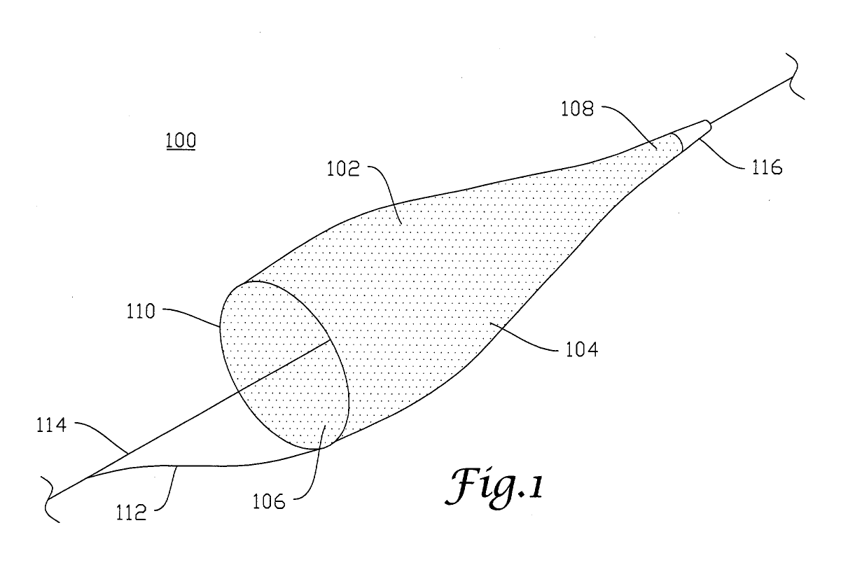

FIG. 1 is a perspective view of an example embolic protection filter device 100, which includes a filter membrane 102. Filter membrane 102 may be formed from any suitable blow moldable material or combination of materials. For example, filter membrane 102 may include polymers such as polyether block amide, polybutylene terephthalate/polybutylene oxide copolymers sold under the Hytrel and Arnitel trademarks, Nylon 11, Nylon 12, polyurethane, polyethylene terephthalate, polyvinyl chloride, polyethylene naphthalene dicarboxylate, olefin/ionomer copolymers, polybutylene terephthalate, polyethylene naphthalate, ethylene terephthalate, butylene terephthalate, ethylene naphthalate copolymers, polyamides, aromatic polyamides, polyurethanes, aromatic polyisocyanates, polyetheretherketone, polycarbonates, polyamide/polyether/polyester, polyamide/polyether, and polyester/polyether block copolymers, among others. Filter membrane 102 is porous, having pores 104 of a size suitable to allow the passage of blood while retaining embolic material of a desired size. Filter membrane 102 has a mouth 106 and a closed end 108 and is capable of moving between an open state and a closed state. Mouth 106 is generally sized to occlude the lumen of the body vessel in which the filter may be installed, thereby directing all fluid and any emboli through the filter.

A flexible hoop 110 may be attached to filter membrane 102 at or proximal to mouth 106. Flexible hoop 110 may be attached to filter membrane 102 through melt bonding or other suitable means. Flexible hoop 110 has an expanded state and a compressed state, the expanded state urging mouth 106 to its full size, and the compressed state permitting insertion into a small lumen. Flexible hoop 110 may be made from a flexible metal such as spring steel, from a super-elastic elastic material such as a suitable nickel-titanium alloy, or from other suitable material. Flexible hoop 110 may be a closed hoop made from a wire of uniform diameter, it may be a closed hoop made from a wire having a portion with a smaller diameter, it may be an open hoop having a gap, or it may have another suitable configuration. A strut 112 may be fixedly or slideably attached to and extend from flexible hoop 110. An elongate member 114 may be attached to and extend from strut 112. Elongate member may be attached to strut 112 at an angle or strut 112 may have a small bend, either at a point or over a region. Strut 112 may be attached to hoop 110 at a slight angle such that when elongate member 114, strut 112, and hoop 110 are in an unconstrained position, elongate member 114 may generally extend perpendicular to hoop 110. In the unconstrained position, elongate member 114 may also lie along an axis which passes through the center of the region created by hoop 110. This may help position hoop 110 in contact with the wall of a vascular lumen or it may help in enhancing predictability or reliability during deployment. Elongate member 114 may terminate at strut 112 or it may extend through filtering membrane 102, as shown. Whether or not elongate member 114 extends through filtering membrane 102, it may be fixedly or slideably/rotatably attached to the membrane. Filter membrane 102 may include waist 116 at closed end 108. Waist 116 may be integrally formed with filter membrane 102. Integrally forming waist 116 with filter membrane 102 may reduce the outer diameter of the filter device when in a compressed state, increase the reliability and uniformity of the bond between the filter membrane and the elongate member, and reduce the number of steps or components needed to form the filter device. Waist 116 is a region incapable of moving between two states and having a lumen of substantially constant diameter therethrough. Elongate member 114 may extend through and be bonded to waist 116. This bonding may be heat bonding such as laser bonding or may be an adhesive or other suitable means.

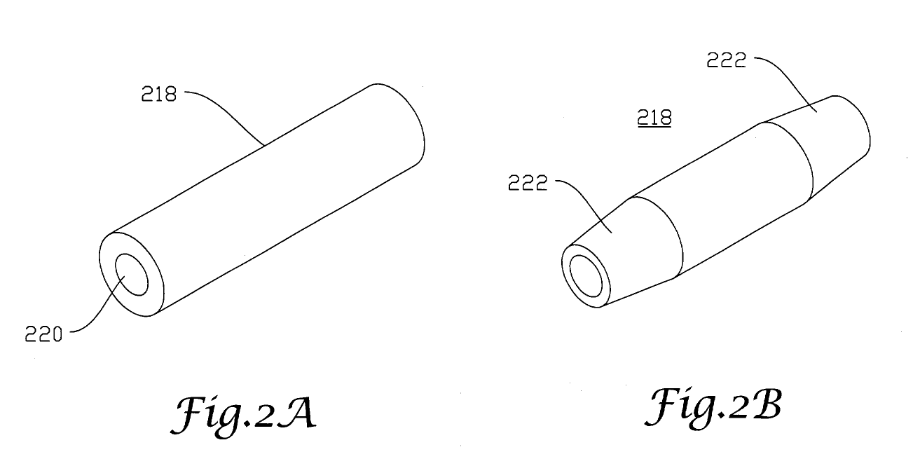

FIG. 2a is a perspective view of an example polymer tube 218 suitable for use in making an embolic protection filter. Tube 218 has a lumen 220 extending therethrough and may comprise polymers such as those listed above with reference to FIG. 1. Tube 218 may be extruded or fashioned using another suitable process. The use of tube 218 will be discussed in detail below.

FIG. 2b is a perspective view of another example polymer tube 218 suitable for use in making an embolic protection filter. Tube 218 includes a non-uniform outer surface 222, which surface may enhance certain characteristics of a filter membrane manufactured therefrom such as thickness and uniformity. This non-uniform outer surface may include narrowing end portions, as shown, or it may include other suitable shapes and configurations. For example, narrowing end portions may permit integrally formed waists to be formed that have a reduced outer diameter. Other suitable polymer tubes may include a non-uniform inner surface.

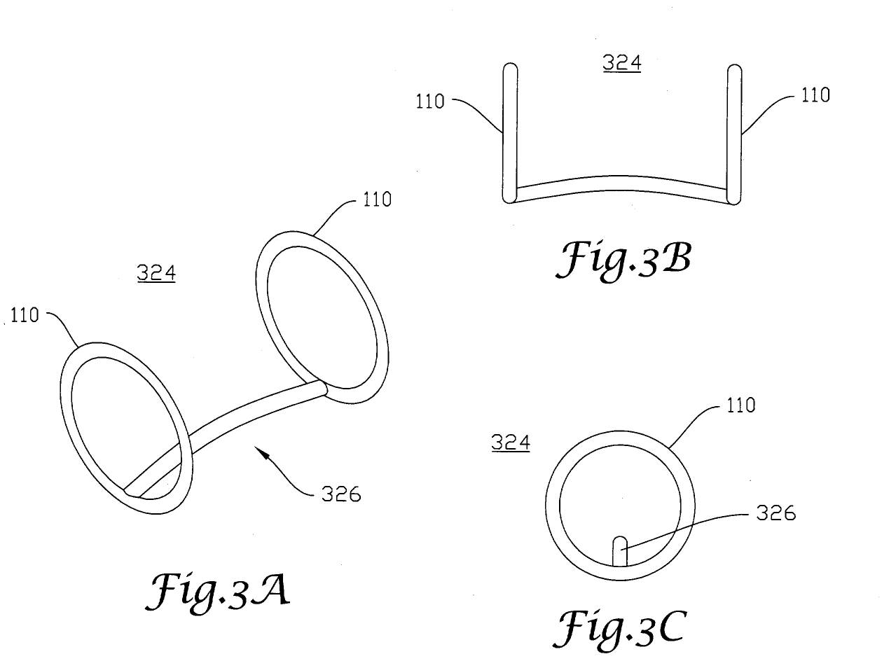

FIG. 3a is a perspective view of an example flexible hoop and strut apparatus 324 suitable for use in making one or more embolic protection filter devices. FIG. 3b is a front view of apparatus 324 and FIG. 3c is an end view of apparatus 324. Apparatus 324 includes two flexible hoops 110 connected by one or more elongate members 326. For instance, apparatus 324 may include one elongate member, as shown, or may include two, three or more elongate members, as may be desired. Elongate members 326 may include one or more struts 112, each strut 112 attached to a flexible hoop 110. The struts may be attached to the hoop though laser welding, soldering, or other suitable means. Thus, apparatus 324 may be separated into two strut and flexible hoop assemblies, if desired. Alternatively, one or more struts 112 may extend from each flexible hoop. Having the two strut and hoop assemblies joined in apparatus 324 may enhance the ease of positioning the strut and hoop assembly in a molding apparatus and may permit two filter devices to be formed simultaneously, as described below.

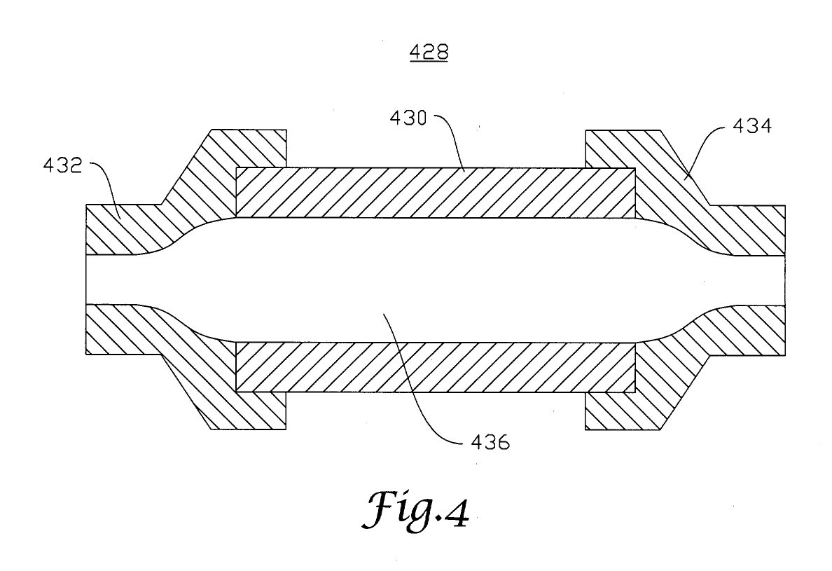

FIG. 4 is a front section view of a portion of a blow molding apparatus 428 suitable for use in making one or more embolic filter protection devices. The blow molding apparatus includes center portion 430, end portion 432 and end portion 434. When assembled together, the portions 430, 432, and 434 define a cavity 436 which may have a desired profile for a filter membrane or two filter membranes. If the cavity is suitable for the forming of two filter membranes simultaneously, the regions of cavity 436 which define the waist or narrow end of the filter membrane will be farthest from each other and there may be a region between those portions of the cavity which have a filter membrane profile which does not have a profile used to define a filter membrane. This region may coextend with the region between hoops 110 of apparatus 324.

An example embolic filter protection device 100 may be manufactured according to the following method. A polymer tube may be extruded having one or more layers and a central lumen. The tube may then be stretched, with or without a fluid such as air in the central lumen, to at least partially orient the polymer. The tube may be modified to vary the outer diameter and/or the inner diameter as desired using a suitable technique described below. It may be desirable to keep the moisture content of the tube low, for example, below 0.15%. This may be done by drying the tube at a low temperature, removing moisture from the surrounding atmosphere or applying a desiccant.

The outer diameter of the tube may be modified by removing material from the outside of the tube. This may be done using, for example, centerless grinding or chemical etching. The inner and outer diameters of the tube may be modified by using a selective stretching technique. In one example technique, a portion of the tube such as the center portion of the tube is kept at or below the glass transition temperature of the material comprising the tube while the portions to be modified are kept at a higher temperature. The tube is then stretched. The portions to be modified will undergo a reduction of inner and outer diameters as well as a lengthening. It may be desirable to keep the tube under tension while cooling it to maintain the deformation.

In another example technique, the portion of the tube to be stretched is selectively secured, for example as by clamping the ends of that portion, and stretched. If desired, this may be done while in a blow molding apparatus.

The tube, if not already in a blow molding apparatus, is then inserted into a blow molding apparatus. Portions of a suitable apparatus are shown in FIG. 4. It may be desirable to pretension the tube prior to molding. The tube is then blow molded by heating and applying a pressure in the lumen of the tube, resulting in radial expansion of the tube to the limits of the blow molding cavity. It may be desirable to maintain tension on the tube while cooling it after the molding process. The tube may be further stretched after blow molding to reduce the inner and outer diameters of the waist portions.

Alternatively, while in the blow molding apparatus, the tube may be exposed to a series of pressures while portions of the tube are exposed to elevated temperatures. For example, a first portion of the blow molding apparatus and tube are dipped into a hot water bath and then exposed to a first pressure. The tube is then further dipped into the hot water bath and then exposed to a second pressure. Finally, the tube is further dipped into the hot water bath and exposed to a third pressure, which may be the same as the first pressure. The tube may be quenched by exposure to a cool water bath.

The molded tube may be turned into a filtering membrane by use of a suitable technique such as laser drilling, mechanical perforation, or chemical etching, or a combination of one of these techniques with annealing to produce pores of the desired size.

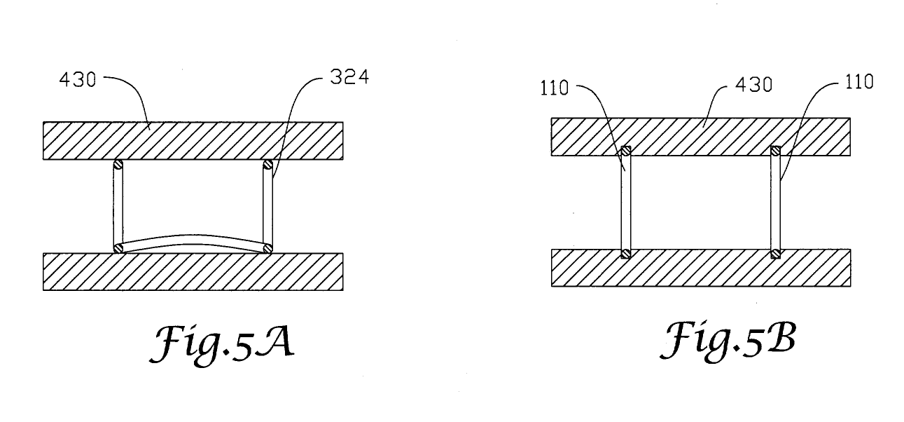

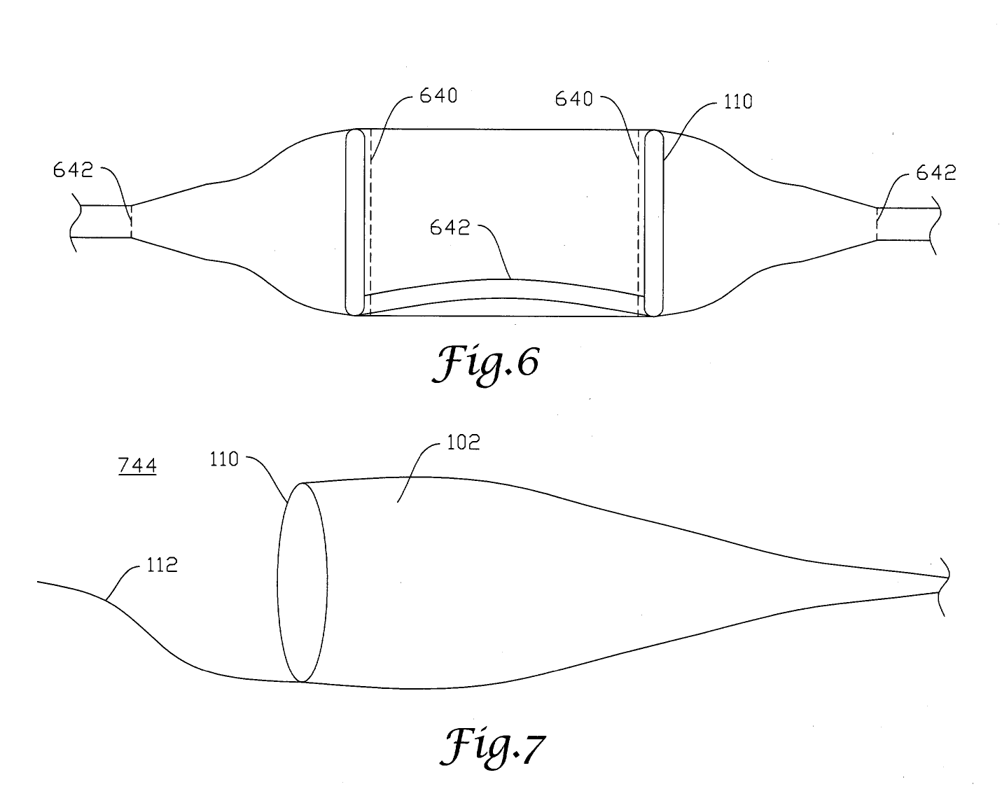

It is contemplated the blow molding process and the assembly process may be simultaneous. Hoops or a hoop and strut apparatus such as that depicted in FIG. 3 may be installed into the blow molding apparatus prior to the blow molding process as shown in FIG. 5A. The hoops or a hoop and strut apparatus may be held in place by a minor interference fit, adhesive, grooves in the blow molding apparatus as shown in FIG. 5B, or other suitable means. The hoop and strut apparatus may include a polymeric or other tie layer on the hoop to aid in forming a bond between the hoop and strut apparatus and the tube. During the blow molding process, both the tube and the hoop and strut apparatus are heated. When the pressure pushes the tube wall against the hoop, a bond may be formed. A plurality of holes may be formed in the tube, as described above. As shown in FIG. 6, the filter membrane may then be trimmed at points 640 proximal hoops 110 and the struts may be trimmed at point 642 to produce one or more hoop, strut and filter membrane assemblies 744, which may then be attached to elongate members, as desired to produce embolic protection filter devices. It can be seen that in the configuration shown in FIG. 6 that two filter devices will be formed through this method.

If the molding process and the assembly process are not simultaneous, the molded tube may be trimmed to produce one or more shaped filter membranes which may be joined to a hoop using heat bonding, adhesive or other suitable means.

It may also be desirable to attach an elongate member to the device. The elongate member may be attached to the strut through welding, adhesive or other suitable technique. The elongate member may also be extended through the lumen in the waist and then attached to the waist through laser or heat bonding or other suitable technique. If it is not desired to attach an elongate member to the waist, the lumen in the waist may be sealed shut using crimping, heat sealing, or other suitable technique.

Of course, while these techniques have been described with respect to blow molding, it is contemplated that many of these techniques have equal applicability to other fabrication methods, such as vacuum molding.

What is claimed is:

1. An embolic protection filter, comprising:
a flexible hoop; and
a filtering membrane melt-bonded to the flexible hoop at a first end.

2. The filter of claim 1, wherein the filtering membrane substantially encapsulates the flexible hoop.

3. The filter of claim 1, further comprising a polymeric tie layer between the filtering membrane and the flexible hoop.

4. The filter of claim 1, wherein the flexible hoop defines a lumen.

5. The filter of claim 4, wherein the flexible hoop includes a circumferential gap.

6. The filter of claim 1, wherein the flexible hoop comprises a shape memory alloy.

7. The filter of claim 1, wherein the filtering membrane defines a cavity having a lumen at the first end.

8. The filter of claim 1, wherein the filtering membrane comprises oriented polymer.

9. The filter of claim 1, wherein the filtering membrane comprises a polymer selected from the group consisting of polyether block amide, olefin/ionomer copolymers, nylon, polyurethane, polyethylene terephthalate, polyvinyl chloride, polyethylene naphthalene dicarboxylate and mixtures or copolymers thereof.

10. The filter of claim 1, wherein the filtering membrane has a thickness of between 0.0006″ and 0.0009″.

11. The filter of claim 1, wherein the filtering membrane has a thickness of between 0.0003″ and 0.0006″.

12. An embolic protection filter, comprising:
a flexible hoop;
a filtering membrane having a first end and a second end and a lumen extending therebetween, the filtering membrane disposed on the wire frame at the first end, and the filtering membrane being movable between an expanded position and a collapsed position; and
a waist integrally formed with the filtering membrane at the second end, the waist having a first end, a second end and a lumen extending therebetween.

13. The embolic protection filter of claim 12, further comprising an elongate member disposed longitudinally through the filter membrane and through the distal waist.

14. The embolic protection filter of claim 12, wherein the elongate member is heat bonded to the distal waist.

15. The embolic protection filter of claim 12, wherein the elongate member is laser bonded to the distal waist.

16. The embolic protection filter of claim 12, wherein the filtering membrane comprises an oriented polymer.

17. A method of manufacturing a filter, the method comprising the steps of:
providing a moldable tube having an outer surface and an inner surface;
providing a blow molding apparatus;
positioning the moldable tube in the blow molding apparatus;
blow molding the tube to form a membrane movable between a first expanded position and a second contracted position; and
forming a plurality of holes in the membrane to form a filtering membrane.

18. The method of claim 17, further comprising the step of shaping the outer surface of the tube.

19. The method of claim 17, wherein the blow molding apparatus includes a mold cavity having a region corresponding to the desired profile of the filtering membrane in an expanded position, and further comprising the steps of:
positioning a flexible hoop in the mold cavity; and
affixing the flexible hoop to the membrane during blowing.

20. The method of claim 19, further comprising the step of heating the flexible hoop.

21. The method of claim 19, further comprising the step of pre-encapsulating the flexible hoop with polymer.

22. The method of claim 19, further comprising the step of providing a tie layer between the flexible hoop and the polymer.

23. The method of claim 19, further comprising the step of providing means in the mold cavity to secure the flexible hoop.

24. The method of claim 23, wherein the means comprises a notch in the mold cavity.

25. The method of claim 23, wherein the means comprises a temporary adhesive selectively applied between the flexible hoop and the mold cavity.

26. The method of claim 23, wherein the means comprises additional shafts affixed to the flexible hoop.

27. The method of claim 17, further comprising the step of trimming the filtering membrane.

28. The method of claim 17, further comprising the step of blow molding the tube to form a waist integrally formed with the membrane.

29. The method of claim 28, further comprising the step of providing a lubricious layer on at least a portion of the inner surface of the tube.

30. The method of claim 28, further comprising the steps of:
providing an elongate member; and
heat welding at least a portion of the waist to the elongate member.

31. The method of claim 28, further comprising the step of laser welding at least a portion of the waist to the elongate member.

32. The method of claim 17, further comprising the steps of:
providing a strut;
providing an elongate member;
attaching the strut to the flexible hoop and to the elongate member.

33. A method of making two filters concurrently, the steps comprising:
providing a moldable tube having an inner surface and an outer surface;
providing a blow molding apparatus;
positioning the moldable tube in the blow molding apparatus;
blow molding the tube to form a membrane;
forming a plurality of holes in the membrane to form a porous membrane; and
removing excess material from the porous membrane to form a first filtering membrane and a second filtering membrane.

34. The method of claim 33, further comprising the step of providing a mold cavity having a first interior region corresponding in shape to the desired profile of a first filtering membrane and a second interior region corresponding in shape to the desired profile of a second filtering membrane.

35. The method of claim 34, wherein the first interior region and the second interior region are longitudinally aligned.

36. The method of claim 35, wherein the first interior region and the second interior region each have a first end and a second end, wherein the transverse cross sectional area of the first ends is less than the transverse cross sectional area of the second ends, and wherein the second ends of the first interior region and the second interior region face each other.

37. The method of claim 34, further comprising the steps of:
providing a first and a second flexible hoop; and
positioning the first and the second flexible hoops in the mold cavity.

38. The method of claim 37, further comprising the steps of:
providing a first strut having a first end attached to the first flexible hoop and a second end;
providing a second strut having a first end attached to the second flexible hoop and a second end; and
providing an attachment between the second end of the first strut and the second end of the second strut.

39. The method of claim 37, further comprising the step of providing two or more elongate members, each elongate member having a first end attached to the first flexible hoop and a second end attached to the second flexible hoop.

40. The method of claim 39, wherein the step of providing two or more elongate members includes the step of configuring the two or more elongate members to receive the moldable tube to position the first and the second flexible hoops with respect to the moldable tube.

41. The method of claim 40, wherein the moldable tube includes an indentation in the outer surface.
